(19) United States
(12) Patent Application Publication
(10) Pub. No.: US 2011/0232344 A1
(43) Pub. Date: Sep. 29, 2011

(54) CONCENTRATION OF ALGAL BIOMASS

(75) Inventors: Ian James Miller, Lower Hutt (NZ); Rhys Antony Batchelor, Palmerston North (NZ)

(73) Assignee: AQUAFLOW BIONOMIC CORPORATION LIMITED, (NZ)

(21) Appl. No.: 13/063,380

(22) PCT Filed: Sep. 11, 2009
(86) PCT No.: PCT/NZ09/00193

§ 371 Date: Jun. 10, 2011

Related U.S. Application Data
(60) Provisional application No. 61/096,276, filed on Sep. 11, 2008.

(51) Int. Cl. C05F 11/00; C12N 1/12; C10L 1/00; A23K 1/00

(52) U.S. Cl. 71/11; 435/257.1; 44/300; 426/61

(57) ABSTRACT

A method for producing an algal biomass that readily separable from water, and preferably sterile, the method comprising heating an aqueous slurry of algae comprising a mixture of an algal biomass, optionally together with a suitable separation agent, and water in a pressure vessel at a temperature of about 140° C. to about 300° C. and a pressure sufficient to maintain the liquid phase. The method produces an algal biomass that is more readily separable from water and an aqueous phase containing organic chemicals.

### FIELD OF THE INVENTION

The present invention relates to a process for readily separating sterilized microalgal biomass from water, or for concentrating algal biomass in water, and at the same time, to recover some nitrogenous material in the form of valuable organic chemicals, so that the microalgae are more readily recovered as a solid for drying and usage, e.g. for stock food, or for preparing a more concentrated dispersion in water for subsequent manufacture of chemicals or biofuels.

### BACKGROUND TO THE INVENTION

Currently, virtually all transport fuels and most of the carbon-based products of the chemical industry come from oil. Recently, it has become apparent that such oil supplies are limited, and a replacement source of such fuels and chemicals will be required. While various proposals have been made for electric-powered transport, it seems highly likely that a high demand for liquid fuels will continue into the immediate future. Further, there is no replacement for many of the materials dependent on organic chemicals. Accordingly, there is considerable need to find different sources for these materials.

Similarly, most synthetic nitrogenous fertilizer is made from synthesis gas which, in turn, is made from oil refineries or natural gas, and as these sources run down, replacements will be required. There is a potential similar problem with phosphate fertilizer. There are limited sources of concentrated phosphates remaining, and eventually these deposits will become exhausted. In each case, of course, the use of these fertilizers leads to a further problem, in that once applied, the fertilizer is washed away and eventually ends up in river systems, lakes and the sea, often giving rise to unfortunate pollution.

A further problem facing the world is to provide an increasing population with a reasonable and cheap high protein food component. Birds such as chickens may provide a solution, however they also need feeding, and grain feed for chickens competes with human food when stocks are limited.

One possible part-solution to such problems are microalgae, which are perhaps the most rapidly growing plants on Earth. Microalgae have an unusual internal chemistry amongst plants in that they appear to use lipids as reserve energy storage materials, and since they do not have large-scale structures, instead of producing carbohydrates they produce lipid acids for reserve energy storage, which is highly desirable for the production of biofuels. They also make a considerable amount of protein, hence microalgae could have many uses as supplementary stock food, etc, particularly since they are relatively rich in certain unsaturated lipid acids that are highly desirable. Finally, they grow very rapidly in nutrient-rich water, and can strip the water of nitrogenous compounds and phosphate, hence they are useful for producing clean water from polluted water. Even more helpfully, microalgae will grow adventitiously in waste-water, they reproduce rapidly, and so long as sufficient time is given to them, they will remove essentially all the nitrogenous matter and phosphates from the water.

Despite these advantages, very little commercial use has been made of microalgae, and the reason is that while they are very easy to grow, they are very difficult to harvest. Thus while it is reasonably straight forward to isolate microalgae as a 5% concentration in water, it is more difficult to enrich this to 10%, and it becomes increasingly more difficult, or energy intensive, to concentrate them further. Also, even if the algae are dried, if a concentrated aqueous dispersion is required (as would be the case for hydrothermal processing) on rehydrating, smooth dispersions with concentrations of greater than 10% are very difficult to make and accordingly if water is required in subsequent processing, it is very difficult to avoid heating vast amounts of water, which may be inefficient in terms of both energy and capital utilization of processing plant.

Furthermore, if microalgae grown in sewage treatment plants are dried, there remains the problem of whether the product is sterile, and there is also the problem that the dried microalgae have an unpleasant smell. In short, the product may be both unpleasant and dangerous to handle. On the other hand, if fuels are to be made from microalgae, to be economic large volumes of microalgae have to be processed, which often would require microalgae from several sewage treatment sites to be brought to a central processing site. Accordingly, microalgae must be concentrated, and made safe to handle.

One approach to the problem of producing liquid fuels from microalgae has been to carefully grow special strains of microalgae under controlled conditions, such as in bioreactors, tubes, or between plastic sheets, which carries its own costs, where it is possible to produce microalgae with up to 50 wt % lipid content. Such lipids can be extracted and transesterified, thus producing an equivalent to the biodiesel produced from oilseeds and from tallow. However, the extraction of lipids from wet microalgae is also somewhat difficult to carry out efficiently as many solvents tend to be absorbed by the microalgae, which leads to the formation of emulsions from which it is difficult to separate any phase. Accordingly, the microalgae should be dried, which in turn requires a means of efficiently separating the microalgae from the water.

A further aspect of microalgae is that to survive they have to have a density very close to that of water, to avoid sinking from light, and to avoid floating and forming a scum on the surface of the water. They also have an external layer that is highly water-attractive, which helps them prevent clumping. These features make harvesting the algae quite difficult.

As is known by those practised in the art, any harvesting of algae, even to make a 3% dispersion, generally requires the addition of chemicals such as alum or polyacrylamides, in which case these additives are undesirable for some uses, such as stock food.

Yet a further aspect of microalgae is that their lipids contain a number of fatty acids that are regarded as being exceptionally beneficial to health, such as omega-three acids and certain other polyunsaturated acids. The source of these is currently restricted, yet while microalgae offer in principle a very large source, obtaining such fatty acids free of undesirable contaminants is a problem that appears to have prevented this resource from being utilized.

To date, for these and other reasons the successful development of a commercial process for producing microalgae has not been achieved. There remains a need to provide such a process or to at least go some way towards providing such a process.

It is therefore an object of the present invention to provide an improved or alternative method for processing algal biomass so that it is more readily separable from water, to provide a method of producing a biomass fraction that retains as much of its original nature as possible but is free of at least one of the foregoing problems, or to at least provide the public with a useful choice.

### SUMMARY OF THE INVENTION

In a first aspect, the present invention relates to a method for producing an algal biomass that is readily separable from water, and preferably sterile, the method comprising heating an aqueous slurry comprising an algal biomass, optionally a separation agent, and water in a pressure vessel at a temperature of about 140° C. to about 300° C. and at a pressure that maintains the water in the liquid phase. The method produces an algal biomass that is more readily separable from water and an aqueous phase containing organic chemicals.

In one embodiment the method comprises heating a mixture of an algal biomass, a metal oxide or hydroxide and water in a pressure vessel at a temperature of about 150° C. to about 250° C., preferably about 150 to about 200° C., and at a pressure that maintains the water in the liquid phase.

In one embodiment, the separation agent is a metal oxide or hydroxide.

In a further embodiment, the metal oxide or hydroxide is an oxide or hydroxide of magnesium, calcium, strontium, barium, zinc or cadmium, or any combination of any two or more thereof.

In one embodiment the aqueous slurry comprises about 1 to about 80% by weight algal biomass.

In one embodiment the aqueous slurry comprises 1 to about 30% by weight separation agent.

In one embodiment the aqueous slurry is heated at an autogenous pressure, such that the aqueous slurry is maintained in the liquid phase. In various embodiments the biomass is heated at a pressure of about 0.1 to about 35 MPa, about 0.1 to about 9 MPa, or about 0.1 to about 8.59 MPa.

In one embodiment the aqueous slurry is heated for about 1 to about 300 or about 5 to about 300 minutes or more.

In one embodiment the method further comprises concentrating the algal biomass such as by separating the heated algal biomass from some or all of the water to produce a concentrated aqueous dispersion comprising a mixture of algal biomass and water. In one embodiment, the algal biomass is concentrated or separated from the water by filtration, by centrifugation or by settling. In another embodiment, the algal biomass is concentrated or separated from the water by flotation or by decanting.

In various embodiments, following concentration or separation the concentrated aqueous slurry comprises at least about 30 to 99% by weight algal biomass.

In one embodiment, the concentrated or separated microalgae is subjected to further processing to produce a biofuel, a biofuel precursor, fatty acids or one or more organic chemical products.

In one embodiment the concentrated aqueous dispersion is heated in water to supercritical temperatures. This treatment of the concentrated aqueous dispersion is to produce hydrocarbons that, following separation by distillation, give high octane petrol and high cetane diesel biofuels essentially free of nitrogenous material.

In one embodiment the aqueous phase, optionally following extraction to remove nitrogenous material, is heated to supercritical temperatures. This step is to produce mixtures containing hydrocarbons and lactams that, following separation by distillation, give high octane petrol and high cetane diesel biofuels essentially free of nitrogenous material and lactams suitable for use as highly polar solvents.

In one embodiment the concentrated aqueous dispersion is treated with acid to recover fatty acids essentially free of nitrogenous material. Alternatively, solids obtained from the concentrated aqueous dispersion are treated with acid to recover fatty acids essentially free of nitrogenous material. Solids may be obtained by further dewatering such as drying.

In one embodiment the concentrated aqueous dispersion is used as stock feed. In one embodiment, the concentrated or separated microalgae is available as a potential animal feed. Alternatively, solids obtained from the concentrated aqueous dispersion are used, preferably after drying.

In one embodiment concentrated aqueous dispersion is used as fertilizer. In one embodiment, the concentrated or separated microalgae is available as a nitrogen and phosphate-rich fertilizer. Alternatively, solids obtained from the concentrated aqueous dispersion are used, preferably after drying.

In one embodiment, the water from which microalgae has been removed is subjected to further processing to produce a biofuel, a biofuel precursor or one or more organic chemical products.

In another embodiment, the method is a method for producing a concentrated aqueous dispersion of algae, the method comprising

i) heating an algal biomass in a pressure vessel at about 140° C. to about 300° C. in the presence of an organic solvent that is immiscible in water to form an algal biomass and an organic extract that are readily separable from water, and
ii) removing water from the algal biomass that is readily separable from water to produce a concentrated aqueous dispersion of algae.

In one embodiment the method further comprises extracting chemicals from the aqueous phase with an organic solvent.

In one embodiment, the concentrated aqueous dispersion of algae is subjected to further processing to produce a biofuel, a biofuel precursor or one or more organic chemical products.

In another aspect, the present invention relates to an algal biomass that is readily separable from water, produced by a method of the invention.

In another aspect the present invention relates to a concentrated aqueous dispersion of algae, produced by a method of the invention.

In another aspect the invention provides a biofuel, biofuel precursor or one or more organic chemical products produced by a method of the invention.

In one embodiment of any of the above aspects where an organic chemical is produced, the one or more organic chemical products may include oxygenated species such as methylated cyclopent-2-en-1-ones, nitrogen heterocycles including indole, 2-methyl piperidine, N-ethyl piperidine, N-ethyl pyrrole, pyrimidine, methyl pyrazine, dimethyl pyrazine, ethyl pyrazine, 2-ethyl-3-methyl pyrazine, trimethyl pyrazine, 2-ethyl-3,6-dimethyl pyrazine, 2-pyrrolidinone, 2-piperidinone, N-methyl-2-pyrrolidinone, N-ethyl-2-pyrrolidinone, N-butyl-2-pyrrolidinone, 3,6-dilsobutyl-2,5-piperazinedione and other products including lipids and lipid derived compounds including lipid acids, deaminated amino acids such as propionic acid, butanoic acid, methyl butanoic acids, 4-methyl pentanoic acid, and hydrocarbons.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Further aspects and advantages of the present invention will become apparent from the ensuing description which is given by way of example only.

### DETAILED DESCRIPTION OF THE INVENTION

The invention generally relates to a method of heating microalgae, or a mixture of algae and water under pressure, preferably in the presence of certain metal oxides that can react with carboxylic acids to form insoluble carboxylates, to make the algae:

(a) more easily separable from water and/or
(b) more suitable for the extraction or production of fuels and chemicals,
(c) more suitable for use as an animal feed or fertilizer.

Thus a general process for aiding the separation of algae from water comprises heating an aqueous slurry of microalgae as described herein, cooling the slurry, and separating the algal biomass that is readily separable from the water by any method known in the art, such as settling, filtration or centrifugation, to produce either essentially damp algae or to retain some water to make a concentrated aqueous dispersion of algae. If the objective is to make a more concentrated dispersion, then settling and decanting off surplus water is often more desirable. Adding algae back to less water may be useful to further process the algae into a biofuel or biofuel precursor, or to be otherwise processed free of the nitrogen-containing heterocycles removed in the aqueous phase, while the algae-free water can be further processed to recover dissolved organic chemicals.

An embodiment comprises treating the microalgae with metal salts, such as alum, that may be adsorbed by the surface, thus altering the density of the microalgae and assisting in their settling.

One particular problem with filtering microalgae is that the outer walls of the microalgae are soft and deformable, and hence clog filters and make filtration extremely difficult. A second general aspect of this process comprises treating an aqueous slurry of microalgae, as described above, in the presence of certain metal oxides that harden the microalgae, thus making the algae far more easy to filter and settle more quickly, thus more readily separating the solids from the aqueous phase.

Either phase may be further hydrothermally processed, or it may be extracted with a solvent immiscible in water in order to extract organic materials either as separate phases or combined, and either as obtained, or following treatments with acid or base. Suitable water immiscible solvents include, but are not restricted to, methylene chloride and other halogenated hydrocarbons, toluene and other aromatic hydrocarbons, petroleum spirit, esters and ethers. The extracted chemicals are then either isolated, or reacted in the solvent as described below. The residual aqueous fraction may also be further hydrothermally processed.

### 1. Definitions

When a chemical compound is named in the singular, it refers to that specific compound, thus pyrazine would refer to 1,4-diazabenzene. When the term is used in the plural, it refers to the entire set of structures with that structural element, thus pyrazines would include all molecules with the pyrazine structure, including but not restricted to molecules with any substitution such as methylation or any molecule within which the pyrazine structure can be found. If a statement is made involving such a set of molecules, such as the term pyrroles, the subsequent use of a specific molecule that is an element of that set of molecules, such as indole, does not in any way contradict the generality of the previous statement, but should be taken as a special example or a special case.

The term “algal biomass” as used in this specification means any composition comprising microalgae. The algal biomass may be partially de-watered, i.e. some of the water has been removed during the process used to harvest the algae, for example during aggregation, centrifugation, micro-screening, filtration, drying or other unit operation.

The term “clay” as used in this specification includes any finely-divided aluminosilicate or magnesiosilicate as well as related materials normally termed clays, whether these are of mineral origin or synthesized by some other method.

The term “comprising” as used in this specification means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

The term “pressure vessel” as used in this specification means a container that is capable of holding a liquid, vapour, or gas at a different pressure than the prevailing atmospheric pressure at the location of the pressure vessel.

The term “protein-containing material” means any material that contains protein and it usually also implies that the material also contains lipids. The material will be of biological origin, and apart from microalgae, usually of animal origin.

The term “separation agent” refers to any agent that results in an algal biomass that is readily separable from water when the agent is incorporated into an aqueous slurry comprising algal biomass and heated as described herein.

The term “settling” as used in this specification means the process by which microalgae proceed to the bottom of the fluid in which they are suspended due to gravity, or down the potential field of any corresponding inertial force, such as occurs in a centrifuge.

The term “stock” as used in this specification means any animal that is kept and fed by humans, and can eat microalgae. “Stock food” is thus any food fed to such animals, which may include mammals such as cows, birds such as chickens, farmed fish, shellfish, or any other member of the animal kingdom.

“Wastewater” includes water that has been used for some purpose and consequently requires further treatment. It may refer to fresh or saline water, effluent from sewage treatment plants and water from facilities in which domestic or industrial sewage or foul water is treated.

### 2. Feed Materials

The algal biomass for use in the process of the invention comprises single-cell micro-algae. In one embodiment the algae are algal species that are naturally present in the local environment and that grow in the water without seeding. In another embodiment the algae are algae species that have been specifically seeded in a pond. In another embodiment the micro-algal biomass is harvested from wastewater.

Algae of use in the methods of the invention may be either to mixed species, or specifically grown monocultures. While the microalgae generally used here are freshwater members of the Chlorophyta, the scope of the invention is not restricted to this, and the invention also applies to single cell members of other microalgae or cyanobacteria, for example, of the Rhodophyta, and for algae living in seawater. An example of the latter type of microalgae is Dunaliella salina, which can live in extremely salty water. Examples of suitable microalgae include but are not limited to microalgae of Division Cyanophyta (cyanobacteria), microalgae of Division Chlorophyta (green algae), microalgae of Division Rhodophyta (red algae), microalgae of the Division Chrysophyta (yellow green and brown-green algae) that includes the Class Bacillariophyceae (diatoms), microalgae of Division Pyrrophyta (dinoflagellates), and microalgae of Division Euglenophyta (euglenoids), and combinations thereof. Examples of Chlorophyta include but are not limited to microalgae of the genera Dictyosphaerium, Micractiniumsp, Monoraphidium, Scenedesmus, and Tetraedron, or any two or more thereof. Examples of cyanobacteria include but are not limited to microalgae of the genera Anabena, Aphanizomenon, Aphanocapsa, Merismopedia, Microcystis, Ocillatoria, and Pseudanabaena, or any two or more thereof. Examples of Euglenophyta include but are not limited to Euglena and Phacus. Examples of diatoms include but are not limited to Nitzschia and Cyclotella. Examples of dinoflagellates include but are not limited to Peridinium.

The algal biomass will be in the form of a dilute dispersion in water. In various embodiments, the biomass concentration of the slurry comprises about 1, 2, 3, 4, 5, 6, 7, 8, 9, 10, 15, 20, 25, 30, 35, 40, 45, 50, 55, 60, 65, 70, 75 or 80% by weight and useful ranges may be selected between any of these values (for example, about 1 to about 10, about 1 to about 20, about 1 to about 30, about 1 to about 40, about 1 to about 50, about 1 to about 60, about 1 to about 70, about 1 to about 80, about 10 to about 30, about 10 to about 40, about 10 to about 50, about 10 to about 60, about 10 to about 70, or about 10 to about 80% by weight).

### 3. Heating

The process of the invention includes heating a dispersion of algal biomass, preferably in water with the addition of a divalent metal oxide that is partially soluble in water, cooling the mixture and separating the solids from the aqueous phase. Without being bound by theory, it is believed that heating the algae denatures the protein, changing the properties of the algae such that the water associated with it can be removed and it also partially hydrolyses the lipids and protein. The metal oxides, if present in a form that is at least partially soluble, will form insoluble salts or soaps on the surface of the microlalgae, thus altering the surface by making it harder and more hydrophobic. Heating dry algae simply denatures protein.

Heating the microalgae in water will also extract heterocyclic materials that are chemically unbound to other polymers in the microalgae. These are the materials that give the dried product its foul smell, hence extracting these into the water produces a product that is at least less obnoxious. Heating the microalgae under water also sterilizes the material, killing the pathogens that could otherwise be a problem if the algae were harvested from sewage treatment.

The aqueous slurry containing the biomass may be heated at a temperature of about 140 to about 300° C. The choice of temperature should be determined in part by the method of collection of the solid and the equipment to be used and in part by the intended use. For example, if microalgae are to be used as stock food or fertilizer, a minimal amount of calcium hydroxide or magnesium oxide would be used, together with the lower temperature range. If the stock are laying hens, then increased amounts of calcium may be desirable. As a general rule, the higher the temperature, the more nitrogenous material leaches from the microalgae, however too low a temperature either requires a longer time to get the desired effect, or in the limit, there is insufficient effect.

The temperature to which the mass is heated is preferably of about 150 to about 220° C. At higher temperatures, there is no significant improvement in separability but the mass collected is lower as soluble material leaches into the water.

For example, when microalgae were heated in the presence of small amounts of calcium hydroxide, at 150° C., only a very small amount (<3%) of organic material was transferred to the aqueous layer, yet 50% of the products were lipid derived, 32% were indole, and 11.5% were 2-piperidinone (δ-valerolactam). At 250° C. less than 7% was indole and 7.6% 2-piperidinone, the amount of recovered oil was approximately 5 times higher, however there were also considerable non-volatile organic material in the aqueous phase that was not extractible into organic solvent. This result seems to have occurred because the amount of indole capable of being produced is readily produced at the lower temperatures, hence if the objective is to obtain indole that is readily able to be purified, or to obtain a further stream in a subsequent process that is either free or much freer of indole, then 150° C. may be desirable. While indole is used in the perfumery industry, paradoxically by itself it has quite an obnoxious smell, and removing it from microalgae will make the product more acceptable for other users.

If the material is to be used for stock food, the addition of small amounts of calcium hydroxide and magnesium oxide and temperatures of 150° C. would seem desirable. This settles the microalgae, destroys pathogens, removes much of the indoles (which improves the smell of the product) and retains most of the inherent protein and lipid fractions.

Accordingly, in various embodiments the aqueous slurry may be heated at a temperature of at least about 140, 150, 160, 170, 180, 190, 200, 210, 220, 230, 240, 250, 260, 270, 280, 290, or 300° C., and useful ranges may be selected between any of these values (for example, about 140 to about 200, about 140 to about 210, about 140 to about 220, about 140 to about 230, about 140 to about 240, about 140 to about 250, about 140 to about 260, about 140 to about 270, about 140 to about 280, about 140 to about 290, or about 140 to about 300° C.).

The algal aqueous slurry may be heated for at least about 1 minute, for about 5 minutes to about 5 hours, about 10 minutes to about 60 minutes, or about 15 minutes to about 40 minutes. Because there are many species of microalgae, optimum times must be found by testing the given raw material. Accordingly, in various embodiments the aqueous slurry may be heated for about 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 550, 600, 650 or 700 minutes, and useful ranges may be selected between these values (for example, about 1 to about 60, about 1 to about 120, about 1 to about 180, about 1 to about 240 minutes, about 5 to about 60, about 5 to about 120, about 5 to about 180, about 5 to about 240 minutes, or about 5 to about 300 minutes).

### 4. Pressure Treatment and the Pressure Vessel

In the methods of the invention the pressure is one that maintains the water in the liquid phase. In other words, the pressure is equal to or greater than the vapour pressure of water at the selected temperature.

In preferred embodiments, the pressure which may be used is that which is required to maintain appropriate phases of components in the pressure vessel and which may aid control of the reaction at preferred reaction temperature(s).

Preferably, the process may be carried out in a continuous-flow pressurised reactor. The aqueous slurry comprising algal biomass may be fed into such a reactor, or other types of reactors such as a batch-type reactor or a semi-continuous type reactor, (optionally) together with any other reagents which are to be used.

Following the heating step of the process of the invention, the product stream may optionally be cooled before or after the pressure is released.

In various embodiments the aqueous slurry is heated under autogenous pressure in the pressure vessel. In various embodiments the pressure in the pressure vessel is about 0.1,0.2, 0.3, 0.4, 0.5, 0.6, 0.7, 0.8, 0.9, 1, 2, 3, 4, 5, 6, 7, 8, 8.59, 9, 10, 15, 20, 25, 30 or 35 MPa and useful ranges may be selected between any of these values (for example, about 1 to about 30, about 5 to about 25, about 10 to about 25, about 0.1 to about 9 MPa, or about 0.1 to about 8.59 MPa).

### 5. Separation Agents

In one embodiment a separation agent may be added to the aqueous slurry comprising algal biomass prior to or during heating, or both. The presence of the separation agent alters the surface of the microalgae, thus making them easier to filter or faster to settle.

In one embodiment, the separation agent is a metal oxide or hydroxide, including metal oxide-hydroxides. The metal may be an alkali metal, alkaline earth metal, transition metal, post-transition metal, or metalloid, or any combination of any two or more thereof.

Alternatively, in some embodiments the separation agent may comprise a metal sulphide, metal phosphate, metal complex, or a natural or synthetic mineral. The metal may be an alkali metal, alkaline earth metal, transition metal, post-transition metal, or metalloid, or any combination of any two or more thereof.

In some embodiments, the separation agent may further include a pH-adjusting agent to adjust the pH to a desired range, for example an acidic or an alkaline solution, where useful alkaline solutions include an ammonia solution.

In a various embodiments, the separation agent comprises a metal selected from the group comprising aluminium, barium, beryllium, cadmium, calcium, copper (including copper(I) or copper(II)), iron (including iron(II) or iron(III)), lead, magnesium, molybdenum, nickel, strontium, and zinc, or any combination of any two or more thereof. Useful iron oxides include FeO, Fe₂O₃ and Fe₃O₄).

In one embodiment the metal comprises an alkaline earth metal or transition metal. In particular, the metal comprises magnesium, calcium, strontium, barium, zinc or cadmium, or any combination of any two or more thereof.

In those embodiments where the separation agent comprises a metal oxide or hydroxide, the metal oxide or hydroxide may be selected from, but is not limited to alkali metal hydroxides, alkaline earth metal hydroxides, and transition metal hydroxides. Alternatively, the metal oxide or hydroxide may comprise a metal selected from the group comprising aluminium, barium, beryllium, cadmium, calcium, copper (including copper(I) or copper(II)), iron (including iron(II) or iron(III)), lead, magnesium, molybdenum, nickel, strontium, and zinc, or any combination of any two or more thereof. In one embodiment, the metal comprises an alkaline earth metal or transition metal. In particular, the metal comprises magnesium, calcium, strontium, barium, zinc or cadmium, or any combination of any two or more thereof.

In those embodiments wherein the catalyst comprises a metal complex, the complex may be selected from, but is not limited to, metal complexes including one or more nitric oxide ligands, for example iron(II) complexes with one or more nitric oxide ligands.

The preferred separation agents are bases that are at least partially soluble in water or in the extracts from the microalgae at the selected temperature. Preferred separation agents include calcium hydroxide, zinc oxide, magnesium oxide. Materials such as ferric oxide and aluminium oxide are less soluble, but if they are precipitated onto the algae, they aid settling.

In various embodiments the aqueous slurry comprises at least about 1, 2, 3, 4, 5, 6, 7, 8, 9, 10, 11, 12, 13, 14, 15, 16, 17, 18, 19, 20 21, 22, 23, 24, 25, 26, 27, 28, 29 or 30 by weight of one or more separation agents and useful ranges may be selected between any of these values (for example, about 1 to about 5, about 1 to about 10, about 1 to about 15, about 1 to about 20, about 1 to about 25, or about 1 to about 30% by weight).

### 6. Separation of the Algal Biomass From Water and Further Processing

In one method of the invention a mixture of microalgae in water, with or without separation agent, is heated as described above then cooled to produce a dispersion from which algal biomass is more readily separable from water.

In one embodiment fluid is removed from the dispersion by a process such as decantation to produce a concentrated aqueous dispersion of algae, or a wet solid. Accordingly, n various embodiments, following concentration or separation the concentrated aqueous dispersion or wet solid comprises at least about 30, 35, 40, 45, 50, 55, 60, 65, 70, 75, 80, 85, 90, 95 or 99% by weight algal biomass, and useful ranges may be selected between any of these values (for example, about 30 to about 99, about 35 to about 99, about 40 to about 99, about 45 to about 99, about 50 to about 99, about 55 to about 99, about 60 to about 99, about 65 to about 99, about 70 to about 99, about 75 to about 99, about 80 to about 99, about 85 to about 99, or about 90 to about 99% by weight algal biomass).

The fluid can be removed using any method known in the art, including but not limited to settling, centrifugation and filtration. Alternatively decanting or flotation may be used.

The concentrated aqueous dispersion of algae or the wet solid can be readily dried and stored. This dried product may be used for many applications including as animal feed (for example, chicken feed), or as a stock food supplement, or as a feedstock for further processing.

In one embodiment, the solid residue is subjected to further processing to produce a biofuel, a biofuel precursor or one or more organic chemical products, including fatty acids.

The concentrated aqueous dispersion of algae provides a convenient feed stock for further processing methods, for example, hydrothermal processing under supercritical conditions. Using a concentrated aqueous dispersion of algae as the feed stock for these processes greatly reduces the energy needed to reach supercritical conditions as much less water need be heated. In addition, these processes may now be efficient on a smaller scale using smaller equipment as a higher proportion of algal material will be reacted, relative to the water present.

In another embodiment, the fluid removed from the algal biomass is extracted with organic solvent to produce one or more organic chemical products, or subjected to further heating to produce further chemicals from the water-soluble material.

These organic chemical products are derived from materials that pre-exist in the algae, which are rich in lipids and nitrogen-containing compounds. The lipid acids are converted to insoluble soaps if sufficient suitable metal oxides are added, and these remain in the solids.

Extraction solvents must be immiscible with water. Suitable solvents include but are not limited to methylene chloride and other halogenated hydrocarbons, toluene and other aromatic hydrocarbons, petroleum spirit, esters and ethers.

In another embodiment, the organic material is removed from the fluid by adsorption onto an activated clay, and subsequently removed by distillation. The amount of organic material is relatively small when the reactions have been carried out at lower temperatures

Such heterocyles can be isolated by methods known to those practised in the art, such as extraction, selective extraction (e.g. by acidifying the water to pH <0 pyrroles may be selectively extracted, along with carboxylic acids, which may be removed by base extraction) adsorption, precipitation, etc, and subsequently by fractional distillation, or crystallization.

In the methods of the invention, nitrogen heterocycles and other chemicals can be separated from the remaining organic chemical products obtained by solvent extraction of the aqueous phase. Extracting the aqueous phase at neutral pH leads to the extraction of weakly basic nitrogenous heterocycles, such as pyrazines, pyrimidines and lactams. Carboxylic acids are obtained by acidifying the water to a pH less than 1, and extracting with an organic solvent. The acidic aqueous layer is then separated and made basic then extracted with a solvent to obtain basic amines. The solvent is removed to provide an organic chemical product that is rich in nitrogen heterocycles.

Alternatively, the organic fraction is dissolved in solvent, dried and gaseous strong acid, such as hydrogen chloride, is passed through it. The nitrogen heterocycles precipitate and can be removed by filtration. Specifically, the organic fraction can be treated with any gaseous strong acid including but not limited to hydrogen chloride, hydrogen bromide or hydrogen iodide. The resultant precipitate is recovered by filtration or centrifugation, washed with dry solvent and added to acidified water such that the pH is 0. The solution is extracted with organic solvent to obtain pyrroles and indoles. The solution is then neutralized and extracted with organic solvent, diazines and lactams are extractible, which can subsequently be separated by distillation.

Alternatively, the solution may be made basic initially, and exhaustively extracted to obtain all organic material except that which makes carboxylate anions, these being obtained by making the pH <1 and extracting with solvent.

Alternatively, following the extraction of the basic amines, the solution may be acidified and passed over clays or zeolite catalysts to form aromatic hydrocarbons including toluene, xylene, trimethyl benzene and ethyl benzene.

The nitrogen heterocycles obtained in the process of the invention are commercially valuable. The pyrazines are useful as intermediates in the preparation of pharmaceuticals and cosmetics. The pyrrolidinones and piperidinones are lactams, and hence could be used to make biologically derived nylons, specifically nylon 4 and nylon 5 or used as highly polar high-boiling solvents. While the yields may not be high, if microalgae are to be the basis of a fuels industry, even quite low fractions could be of commercial significance.

In a further variation of the process of the invention, an aqueous slurry of microalgae, concentrated to a level of microalgae that can most conveniently be attained, is heated at temperatures of about 150° C. to about 300° C., or more preferably about 150° C. to about 200° C., holding the temperature there for a period about 1 minute to about five hours, more preferably about 10 minutes to about 45 minutes, then gradually reducing the pressure, adding further heat if necessary, which has the effect of steam distilling the mixture, thus removing the small amounts of volatiles together with more modest amounts of water.

Various aspects of the invention will now be illustrated in non-limiting ways by reference to the following examples.

### EXAMPLES

### 1. Discussion of Examples

If microalgae is dried and then returned to being dispersed in water, the dispersion remains almost as thick and difficult to extract as if it had not been dried, however if the dried microalgae is heated to 150° C., some volatiles are given off and there is a colour change. This process is irreversible, as when the resultant material is dispersed in water, it is no longer extremely hydrophilic and the mixture is more readily extracted with organic solvents, which is desirable if the objective is to extract lipids, or high boiling organic materials.

If microalgae is heated under pressure in water there is a minor improvement in filterability, but there is a marked improvement in settling. Settling is an ideal method for producing a more concentrated dispersion for subsequent processing, however filtration is the most quantitative way of separating solid and liquid.

If heated to 200° C., a little under a half the initial mass is recovered as solid microalgae, and a little over half the initial mass is recovered from solution by evaporating off the water. This solution will initially contain volatiles, polymer fragments such as peptides, and dissolved salts. As the temperature of heating is made higher, both these yields decrease, and the difference is presumed to be the result of forming relatively volatile organic materials that are lost through the heat-accelerated evaporation of the water. Small amounts of volatile organic material were obtained by extraction, and we assume that material lost by evaporation included these materials. On the other hand, if the microalgae are only heated to 150° C. in the absence of metal oxides, there is negligible improvement in filterability.

The clearly identified materials recovered from the process were pyrazines, piperidinediones, deaminated amino acids and basic amines, mainly piperidines. On the other hand, the actual yield of these materials at modest temperatures was low. Upon heating at 250° C., dimethyl disulphide, cyclopentanone and methylated cyclopent-2-en-1-ones formed, while the recovered yields of microalgae were sufficiently small that it was impractical to proceed to higher temperatures if the objective was to concentrate the algae. Accordingly, the solutions run at higher temperatures were not analysed.

Filtration often becomes much simpler if certain chemicals are added. Alum is frequently added to microalgae to assist the formation of filterable precipitates, but we found that alum made little improvement to filterability when the mixtures were heated, although settling ability improved. Some metal oxides or hydroxides did make a significant improvement to filterability, but not all did. In particular, the addition of ferric oxide made no detectable difference, presumably because the oxide is essentially insoluble in water. The same occurred with nickel oxide and copper oxide, although in this case some metallic copper was formed through the reducing conditions present. When good filterability is achieved, adhering liquid can be washed off, which improves the odour of the product.

The addition of calcium hydroxide results in easily filtered solids provided there is 10% calcium hydroxide present. If 5% calcium hydroxide is added, filtration was more awkward, but still possible, while if 2.5% calcium hydroxide was employed, there was no significant difference between it and no addition, although settling ability improved. By heating to 150° C. or higher, however, settling ability is improved, even with small amounts of calcium hydroxide.

The relative conversion of microalgae to volatiles in the presence of calcium hydroxide paralleled that with no separation agent, at least to the extent that the yields of volatiles were similarly dependent on temperature, however there were differences. That the yields of solids was higher simply reflects the fact that calcium hydroxide was also added. Similarly, higher levels of calcium hydroxide increased the yield of non-volatile solids in the aqueous phase, again because of the ease of forming salts. (However, most of the calcium remains in solution, as a counterion for the acids produced by the reaction.) Interestingly, the 20% Ca(OH)₂ reaction at 200° C. gave very few carboxylic acids, even after acidifying, yet there is a high yield of solids in the aqueous phase. This suggests that in this run, deamination was inhibited. Thus if calcium glycinate, say, was formed here, this would provide mass on evaporation, but it would not be extractable following acidification as glycine would be quite soluble in water.

The chemical compounds found in the absence of additives were also found when calcium hydroxide was added, however some new materials also formed, including pyrroles, pyrimidine and the lactams 2-piperidinone and the 2-pyrrolidinones. Benzene propionic acid was also formed, and, at low levels of calcium hydroxide, lipid acid amides were also detected. At higher levels of calcium hydroxide, the calcium soaps of these acids would form, which are expected to be insoluble in water at these temperatures. The aliphatic hydrocarbons were essentially absent, consistent with the calcium soaps being more stable at these temperatures. It was also of interest that 2-pyrrolidinones were only clearly identified from these reactions with calcium hydroxide. However, we emphasize that provided the temperatures are maintained below about 200° C., the yield of such heterocycles was low.

That there was considerable organic material in the aqueous phase that could not be extracted was demonstrated by heating this water to supercritical conditions, which produced a further 2.5 g of material, which was more than was extracted initially. The materials were similar, except that aromatic hydrocarbons were also produced including toluene, xylene and styrene. These are products expected from the supercritical hydrothermal treatment of microalgae. If glycine were converted to a hydrocarbon, less than 20% of the glycine's mass would be recovered, so the lower yields are expected. The products of this reaction also included pyrazines and 2-piperidinone, but also alkylated 2-pyrrolidinones and pyrroles.

A particular advantage of this invention can be seen when the filtrate from a reaction with calcium hydroxide was then reacted hydrothermally with phosphate catalyst at supercritical temperatures. A yield of 22% of oil was obtained, the composition of which was typical of the reactions of microalgae with phosphate at high temperatures. Thus while an amount of organic material was extracted from the microalgae, thus resulting in a reduction in the mass collected, the extracted material could still be converted to fuels and chemicals. The recovered solids could also be treated supercritically to produce oils.

Given that the yield of recovered algae was reasonable at 200° C., but less reasonable at 250° C. with calcium hydroxide, effort was made to determine whether there were any other reagents that would be suitable at 250° C.

The heating of the microalgae in the presence of alum made little improvement to filterability, and little change to the yield of microalgae compared with the absence of additives. The products were also similar to those found in the absence of additives, except that there was an increase in the yields of certain deaminated amino acids, and also 2-piperidinone formed. While there were changes they were not considered to be sufficient to warrant further consideration.

The heating of microalgae in the presence of zinc oxide gave microalgae that was easier to filter even than with the addition of calcium hydroxide, and 5% zinc oxide was approximately equivalent to 10% calcium hydroxide. Zinc oxide was even useful at 2.5% concentration. The aqueous fraction gave chemicals very similar to those obtained from calcium hydroxide solutions.

The heating of microalgae in the presence of sodium carbonate gave no significant improvement to filterability, a lower yield, and no significant improvement to the nature of the chemicals obtained from the aqueous fraction, hence this was not examined further.

Cupric oxide, is quite insoluble in water, but it could be solubilized by amines or ammonia. Accordingly, it was not expected to strongly enhance filterability, and these expectations were met in that when microalogae was heated in the presence of cupric oxide, it did not filter particularly better than without additives. That it had been solubilized by amines or ammonia, however, was shown by the formation of a precipitate of copper, an extreme end to the reactions of reducing aldehydes etc to solutions such as Fehling's solution (cuppramonium hydroxide).

The addition of magnesium oxide was highly effective at enhancing filterability of the algae. However, the use of magnesium oxide also appeared to have the effect of increasing the solubilization of the components of the algae. In this particular example, there was also a significant loss of mass, presumably from highly volatile material that was lost during the evaporation of water.

These experiments are also of interest in that the products, while similar, are not equivalent. In particular, it was found that enhancement of 2-methyl piperidine would be at the expense of 2-piperidinone, which suggests that there is a mechanism by which 2-piperidinone is methylated at the carbonyl group and reduced. Interestingly, the presence of cupric oxide inhibited the production of both the piperidine and the piperidinone.

The relative yield of piperidinone was reduced in the presence of both zinc oxide and magnesium oxide compared with calcium hydroxide, while with magnesium oxide, the production of pyrazines was enhanced, perhaps because a greater fraction of the algae were solubilized.

### 2. General Methodology

300 mL of an aqueous slurry of comprising 6% by weight microalgae and a separation agent or solvent, if required, was placed inside a stainless steel bomb, which was sealed, brought to temperature by placing it in the oven where it was left for 30 minutes, then withdrawn and allowed to cool. The microalgae was filtered and either dried or used for further processing. The aqueous layer was extracted with methylene chloride×2, then acidified and extracted with methylene chloride×2, then made basic and similarly extracted. The methylene chloride extracts were then analysed by gas chromatography coupled to a mass spectrometer, then the solvent was evaporated to obtain an estimated yield of extract.

All gas chromatography was run on a Shimadzu QP2010 plus GCMS with an Rtx-5Sil MS column 30 m 0.25 mm ID with a film thickness of 0.25 mm. The injector temperature was 200° C. The split ratio was set at 10:1 for one minute followed by 10 minutes at 100:1, and then reverting to 10:1. The oven temperature program was as follows; 50° C. for 1 minute then rising at 5 degrees/minute to 100° C., then at 20 degrees a minute to 300° C. and a final hold time of 20 minutes. The gas chromatograph was set to constant velocity mode with a velocity of 30 cm/s. The interface temperature was 250° C. and the ion source was set at 200° C. The detector voltage was set relative to the tuning result. The compounds were identified with the aid of the NISTO5 and NISTO5a compound library databases.

### Example 1

### Dry Algae

50 g of dried microalgae was charged to a round-bottomed flask, which in turn was placed in an oil bath. The algal powder was stirred vigorously, and the oil heated. When the oil temperature reached 150° C., the algae emitted water and organic volatiles, and at the same time the green progressively changed to a darkish grey colour. The temperature was held at 150° C. until the colour change was complete (approximately 10 mins, but this was probably dependent on heat transfer) and the flask was then removed from the bath and allowed to cool. The algae could then be mixed with water in essentially all proportions to give an even dispersion, the fluidity of which depended only on having sufficient water that solid-solid interactions were negligible.

### Example 2

### No Additives

Samples of microalgae in water were heated across a range of temperatures between 200-300° C., the solutions cooled and although the dispersions settled, the solids were recovered by filtration. The yields of solids were: 200° C. (8.11 g), 250° C. (5.3 g), 300° C. (4.67 g). A sample of the solvent was evaporated to dryness, and the solid content of the solutions were 200° C. (9.15 g), 250° C. (7.17g), 300° C. (3 g). The differences would be volatile materials in the aqueous solution, and volatiles within the solids that were lost on drying. The total solids decreased with increasing temperature, which presumably corresponds to the increasing formation of volatiles [200° C. (1.34 g), 250° C. (6.13 g), 300° C. (10.93 g)]. The remaining aqueous solutions were extracted with methylene chloride, then acidified and re-extracted, then made basic and extracted. The components of the extract were:

At 200° C.: aqueous extract, little material, but comprised: n butanoic acid (3.3%), 3-methyl butanoic acid (2.1%), 2-methyl butanoic acid (2.7%), 2-methyl pentanoic acid (7.4%), methyl pyrazine (4.2%), 2,5-dimethyl pyrazine (5.5%), ethyl pyrazine (1.6%), trimethyl pyrazine (5.4%), substituted piperazine diones (>10.5%) and numerous unidentified components. After acidification to pH 1, 0.68 g of material was recovered, which contained butanoic acid (29%), 2-methyl butanoic acid (13.6%), 4-methyl pentanoic acid (7.2%), methyl pyrazine (3.3%), dimethyl pyrazine (1.9%), and numerous unidentified components. After adjusting the pH to 14, 0.12 g of material was recovered, which comprised: N-methyl piperidine (7.5%). 2-methyl piperidine (26.7%). N-ethyl piperidine (6.7%). methyl pyrazine (6.8%), 2,5, dimethyl pyrazine (3.4%), trimethyl pyrazine (2%), and numerous unidentified components.

At 250° C.: aqueous extract, 0.51 g was recovered which comprised: dimethyl disulphide (3%), cyclopentanone (1.2%), methyl pyrazine (13.5%), 2-methyl cyclopent2-en-1-one (4.5%), 2,5-dimethyl pyrazine (18.6%), 2-ethyl-3-methyl pyrazine (2.9%), trimethyl pyrazine (9.9%), 3-ethyl-2,5-dimethyl pyrazine (4.7%), trimethyl hydantoin (1.5%), 3,6-diisobutyl-2,5-piperazinedione (2.5%) and numerous unidentified components. After acidification to pH 1, 0.61 g of material was recovered, which contained butanoic acid (29%), 2-methyl butanoic acid (13.6%), 4-methyl pentanoic acid (7.2%), methyl pyrazine (7.4%), 2,5-dimethyl pyrazine (4.4%), and numerous unidentified components. After adjusting the pH to 14, 0.21 g of material was recovered, which comprised: 2-methyl piperidine (≈15%), methyl pyrazine (7.1%), 2,5-dimethyl pyrazine (6.9%), trimethyl pyrazine (3%), 2-piperidinone (2.1%) and numerous unidentified components.

### Example 3

### The Addition of Calcium Hydroxide

Samples of microalgae (18.6 g/300 mL) were treated with calcium hydroxide and heated across a range of temperatures between 200-300° C. In each case a clean precipitate of microalgae was collected that was easily filtered and was able to be washed. The yields of solids were: 200° C., 10% Ca(OH), (8.05 g); 200° C., 20% Ca(OH), (11.71 g); 250° C. 10% Ca(OH)₂, (9.45 g); 250° C. 5% Ca(OH)₂, (7 g); 300° C., 10% Ca(OH)₂, (12.21 g), A sample of the solvent was evaporated to dryness, and the solid content of the solutions were 200° C., 10% Ca(OH)₂ (8.37 g); 200° C., 20% Ca(OH)₂, (10.64 g); 250° C. 10% Ca(OH)₂, (5.69 g); 250° C. 5% Ca(OH)₂, (7.78 g); 300° C., 10% Ca(OH)₂ (2.63 g). The estimated yield of organic non-volatiles from the aqueous solutions was [200° C., 10% Ca(OH)₂ (8.11 g), 200° C., 20% Ca(OH)₂ (0.5 g), 250° C. 10% Ca(OH)₂, (6.55 g); 250° C. 5% Ca(OH)₂, (7.2 g); 300° C., 10% Ca(OH)₂, (6.44 g)]. For each sample, the aqueous solution was extracted with methylene chloride, then acidified and re-extracted, then made basic and extracted. The components of the extract were:

Treatment of algae at 150° C. with 5% calcium hydroxide gives an aqueous phase with only two significant components: indole and 2-piperidinone, although the yield is sufficiently low to make this result of lesser interest. If the temperature is increased to 250° C., the yield of organic material in the aqueous phase increases by approximately an order of magnitude. Indole and 2-piperidinone remain the major components, although some alcohols related to phytol are also extracted together with extracts as reported below. In principle, these would be satisfactory for making into fuel, however there is an advantage on getting them out here because they are also potentially reactive, being somewhat unsaturated.

At 200° C., 20% calcium hydroxide: the very small aqueous extract contained: pyrrole (2.9%), methyl pyrazine (2.6%), 2,5-dimethyl pyrazine (5.6%), trimethyl pyrazine (4.7%), 2-ethyl-3,6-dimethyl pyrazine (2.8%), 2-piperidinone (4.5%), indole (0.9%), 3,6-diisobutyl-2,5-piperazinedione (2.7%), condensed pyrazines (5.4%) and numerous unidentified components. After acidification to pH 1, 0.14 g of material was recovered, which contained methyl pyrazine (2%), 2,5-dimethyl pyrazine (5.4%), trimethyl pyrazine (4.6%), 2-piperidinone (11.1%), condensed pyrazines (>20%) and numerous unidentified components. After adjusting the pH to 14, 0.13 g of material was recovered, which comprised: methyl pyrazine (0.3%), 2-pyrrolidinone (2.3%), 2-piperidinone (20%) and numerous unidentified components.

At 200° C., 10% calcium hydroxide: aqueous extract, little material, but comprised: 2,5-dimethyl pyrazine (0.6%), trimethyl pyrazine (0.6%), hexadecanamide (2.1%) and numerous unidentified components. After acidification to pH 1, 0.6 g of material was recovered, which contained propanoic acid (4.8%), butanoic acid (25.4%), ethyl pyrazine (0.72%), 2-piperidinone (11.9%), benzene propanoic acid (5.3%) and numerous unidentified components. After adjusting the pH to 14, 0.24 g of material was recovered, which comprised: methyl pyrazine (1.7%), 2,5, dimethyl pyrazine (1.5%), trimethyl pyrazine (0,8%), 2-pyrrolidinone (2.9%), 2-piperidinone (15.4%) and numerous unidentified components.

At 250° C., 10% calcium hydroxide: the aqueous extract gave 0.44 g comprising pyrimidine (3.2%), dimethyl disulphide (1.7%), cyclopentanone (0.7%), 2-methyl cyclopent2-en-1-one (2.1%), methyl pyrazine (13.8%), 2,5-dimethyl pyrazine (10.6%), ethyl pyrazine (8.4%), 2-ethyl-3-methyl pyrazine (4.8%), trimethyl pyrazine (10%), 3,6-diisobutyl-2,5-piperazinedione (3.6%), condensed pyrazines (3.3%) and numerous unidentified components. After acidification to pH 1, 0.88 g of material was recovered, which contained pyrimidine (2.4%), 2-methyl propionic acid (7.4%), butanoic acid (23%), 3-methyl butanoic acid (6.6%), 2-methyl butanoic acid (10.2%), 4-methyl pentanoic acid (12.2%), methyl pyrazine (3.7%), ethyl pyrazine (2.1%), trimethyl pyrazine (0.6%), 2-piperidinone (2.8%), and numerous unidentified components. After adjusting the pH to 14, 0.18 g of material was recovered, which comprised: 2-methyl piperidine (21.3%), methyl pyrazine (7.6%), N-ethyl piperidine (5.8%), 2,5-dimethyl pyrazine (6.7%), trimethyl pyrazine (3.7%), 2-piperidinone (4.2%) and numerous unidentified components.

The treatment of microalgae at 250° C. with 10% Ca(OH)₂ was repeated, leading to 7.7 g solids being recovered, and 6.33 g of non-ashable dissolved solids. When 240 mL of such an aqueous solution was heated to 400° C. for 30 minutes. 1.34 g of oil was recovered, and the aqueous phase retained a further 1.2 g of material. The oil contained toluene (6.1%), xylene (4.9%), styrene (5.6%), cyclopentanone (0.9%), 2-methyl cyclopent2-en-1-one (2.5%), 2,3-dimethyl cyclopent2-en-1-one (1%), N-ethyl pyrrole (2.3%), methyl pyrazine (4.1%), 2,5-dimethyl pyrazine (7%), trimethyl pyrazine (4.3%), N-methyl-2-pyrrolidinone (4.6%), N-ethyl-2-pyrrolidinone (3.7%), tetramethyl pyrrole (0.5%), N-butyl-2-pyrrolidinone and numerous unidentified components. After acidification of the aqueous layer the further recovered material contained acetic acid (12.8%), acetamide (7.1%), n-butanoic acid (10.5%), N-methyl-2-pyrrolidinone (4.4%), 2-pyrrolidinone (5.2%), N-ethyl-2-pyrrolidinone (1.2%), 2-piperidinone (13.2%) and numerous unidentified components.

At 250° C., 5% calcium hydroxide: the aqueous extract gave 0.50 g comprising pyrimidine (1.5%), dimethyl disulphide (2%), cyclopentanone (0.5%), 2-methyl cyclopent2-en-1-one (2.3%), methyl pyrazine (5.9%), 2,5-dimethyl pyrazine (5.6%), ethyl pyrazine (3.8%), 2-ethyl-3-methyl pyrazine (2.9%), trimethyl pyrazine (5.8%), 2-piperidinone (4.8%), 3,6-diisobutyl-2,5-piperazinedione (8.5%), and numerous unidentified components. After acidification to pH 1, 0.59 g of material was recovered, which contained pyrimidine (1.4%), 2-methyl propionic acid (7.9%), butanoic acid (21.6%), 3-methyl butanoic acid (4.9%), 2-methyl butanoic acid (12.8%), 4-methyl pentanoic acid (12.2%), methyl pyrazine (6%), 2,5-dimethyl pyrazine (3.7%), 2-piperidinone (3.1%), and numerous unidentified components. After adjusting the pH to 14, 0.3 g of material was recovered, which comprised: 2-methyl piperidine (14%), methyl pyrazine (9.5%), 2,5-dimethyl pyrazine (3.6%), trimethyl pyrazine (4%), 2-piperidinone (3.5%) and numerous unidentified components.

### Example 4

### The Addition of Alum

Samples of microalgae (18.6 g/300 mL) were treated with alum at 5% and 10% concentrations and heated to 250° C. In each case a precipitate of microalgae was collected that was filtered with difficulty. The yields of solids were: 10% alum (7.1 g); 5% alum, (5.5 g); A sample of the solvent was evaporated to dryness, and the solid content of the solutions for 10% alum was 11.1 g, 5% alum, (0.9 g). The estimated yields of organic non-volatile material from the aqueous solutions were: 10% alum (10 g); 5% alum, (1.8 g). For each sample, the aqueous solution was extracted with methylene chloride, then acidified and re-extracted, then made basic and extracted. The components of the extract were:

At 250° C., 10% alum: the aqueous extract gave 0.31 g comprising pyrimidine (2.3%), dimethyl disulphide (0.4%), cyclopentanone (0.7%), 2-methyl cyclopent2-en-1-one (4.3%), 2,3-dimethyl cyclopent2-en-1-one (0.5%), methyl pyrazine (8.5%), 2,5-dimethyl pyrazine (13.1%), 2-ethyl-3-methyl pyrazine (2.5%), trimethyl pyrazine (7%), 2-ethyl-3,6-dimethyl pyrazine (3.3%), 2-piperidinone (1%), 3,6-diisobutyl-2,5-piperazinedione (5.6%), other condensed piperazinediones (8.2%), condensed pyrazines (35.6%) and numerous unidentified components. After acidification to pH 1, 0.42 g of material was recovered, which contained 2-methyl propionic acid (13.4%), butanoic acid (31%), 3-methyl butanoic acid (3.9%), 2-methyl butanoic acid (19.4%), 2-methyl cyclopent-2-en-1-one (2.2%), methyl pyrazine (2.3%), ethyl pyrazine (1.7%), and numerous unidentified components. After adjusting the pH to 14, 0.23 g of material was recovered, which comprised: 2-methyl piperidine (8.9%), methyl pyrazine (3.1%), N-ethyl piperidine (3.1%), 2,5-dimethyl pyrazine (4.4%), trimethyl pyrazine (2.2%), 2-piperidinone (6.7%) and numerous unidentified components.

At 250° C., 5% alum: the aqueous extract gave 0.46 g comprising pyrimidine (1.8%), 2-methyl cyclopent2-en-1-one (3.4%), methyl pyrazine (8.0%), ethyl pyrazine (6.7%), 2,5-dimethyl pyrazine (6.4%), 2-ethyl-3-methyl pyrazine (2.3%), trimethyl pyrazine (6.1%), 3,6-diisobutyl-2,5-piperazinedione (5.4%), other condensed piperazinediones (1.8%), condensed pyrazinediones (4.6%) and numerous unidentified components. After acidification to pH 1, 0.74 g of material was recovered, which contained 2-methyl propionic acid (9.4%), butanoic acid (23.2%), 3-methyl butanoic acid (5.8%), 2-methyl butanoic acid (12.1%), 2-methyl cyclopent-2-en-1-one (1.9%), methyl pyrazine (2.7%), 2,5-dimethyl pyrazine (1.4%)), 2-piperidinone (3.2%) and numerous unidentified components. After adjusting the pH to 14, 0.1 g of material was recovered, which comprised: 2-methyl piperidine (13.5%), methyl pyrazine (7.5%), N-ethyl piperidine (3.8%), 2,5-dimethyl pyrazine (5.8%), trimethyl pyrazine (3.9%), 2-piperidinone (3.8%) and numerous unidentified components.

### Example 5

### The Precipitation of Aluminium Oxide/Hydroxide

A sample of microalgae (18.6 g/300 mL) was treated with alum at 10% concentration, followed by the addition of sufficient ammonia solution to make the pH equal to 8.4 and heated to 250° C. A precipitate of microalgae was collected that was filtered with difficulty. The yield of solids was 8.9 g. A sample of the solvent was evaporated to dryness, and the solid content of the solutions for 10% alum was 10.6 g. The estimated yield of organic material from the aqueous solution was 9.7 g. The aqueous solution was extracted with methylene chloride, then acidified and re-extracted, then made basic and extracted. The components of the extract were:

The aqueous extract gave 0.38 g comprising pyrimidine (3.2%), 2-methyl cyclopent2-en-1-one (2.5%), methyl pyrazine (9.9%), 2,5-dimethyl pyrazine (18%), trimethyl pyrazine (9.2%), 2-ethyl-3,6-dimethyl pyrazine (4.8%), 2-piperidinone (1.3%), 3,6-diisobutyl-2,5-piperazinedione (2.8%), other condensed piperazinediones (1.2%), condensed pyrazines (2.3%), undecane (1.5%) and numerous unidentified components. After acidification to pH 1, 0.34 g of material was recovered, which contained butanoic acid (32.7%), pentanoic acid (4.8%), 4-methyl pentanoic acid (11.4%), 2,5-dimethyl pyrazine (0.9%), decane (1.2%), undecane (0.4%), 2-piperidinone (1.2%), and numerous unidentified components. After adjusting the pH to 14, 0.08 g of material was recovered, which comprised: methyl pyrazine (4.3%), 2,5-dimethyl pyrazine (3%), trimethyl pyrazine (1.6%), 2-piperidinone (10.4%), xylene (0.8), nonane (1.3%), decane (2.5%), undecane (1.4%), and numerous unidentified components.

### Example 6

### The Addition of Zinc Oxide

Samples of microalgae (18.6 g/300 mL) were treated with different concentrations of zinc oxide and heated to 250° C. In each case a clean precipitate of microalgae was collected that was easily filtered and was able to be washed, with 5% zinc oxide being as good as 10% calcium hydroxide. The yields of solids were: 250° C., 20% ZnO (9.8 g); 10% ZnO, (8.1 g); 5% ZnO, (7.6 g).

A sample of the solvent was evaporated to dryness, and the solid content of the solutions were 20% ZnO (7.1 g); 10% ZnO, (7.5 g); 5% ZnO, (6.5 g), 2.5% ZnO (5.8 g). The estimated yield of water-soluble non-volatile organic material was 20% ZnO (6.4 g); 10% ZnO, (6.7 g); 5% ZnO, (6.0 g), 2.5% ZnO (5.9 g). For each sample, the aqueous solution was extracted with methylene chloride, then acidified and re-extracted, then made basic and extracted. The components of the extract were:

The neutral extract from 20% ZnO gave 0.45 g comprising 2-methyl cyclopent2-en-1-one (3.7%), 2,3-dimethyl cyclopent2-en-1-one (0.4%), methyl pyrazine (8.9%), 2,5-dimethyl pyrazine (14.6%), 2-ethyl-3-methyl pyrazine (3.7%), trimethyl pyrazine (10%), 2-ethyl-3,6-dimethyl pyrazine (5.1%), 2-piperidinone (1.4%), 3,6-diisobutyl-2,5-piperazinedione (4.4%), other condensed piperazinediones (1.6%), condensed pyrazinediones (3.9%) and numerous unidentified components. After acidification to pH 1, 0.49 g of material was recovered, which contained pyrimidine (0.7%), butanoic acid (24.7%), 3-methyl butanoic acid (3.3%), 2-methyl butanoic acid (12.2%), 4-methyl pentanoic acid (12.7%), 2-methyl cyclopent-2-en-1-one (4.2%), methyl pyrazine (4.2%), 2,5-dimethyl pyrazine (3.2%), trimethyl pyrazine (1.5%), 2-piperidinone (1.6%) and numerous unidentified components. After adjusting the pH to 14, 0.23 g of material was recovered, which comprised: pyrimidine (1.4%), 2-methyl piperidine (13.8%), methyl pyrazine (4.4%), 2,5-dimethyl pyrazine (4.5%), 2-ethyl-3-methyl pyrazine (1.4%), trimethyl pyrazine (4.5%), 2-piperidinone (5.8%) and numerous unidentified components.

The neutral extract from 10% ZnO gave 0.44 g comprising pyrimidine (1.2%), cyclopentanone (0.4%), 2-methyl cyclopent-2-en-1-one (2.8%), methyl pyrazine (6.1%), 2,5-dimethyl pyrazine (5.8%), ethyl pyrazine (4.5%), 2-ethyl-3-methyl pyrazine (2.9%), trimethyl pyrazine (6.7%), 2-piperidinone (1.6%), 3,6-diisobutyl-2,5-piperazinedione (10.6%), other condensed piperazinediones (3.2%), condensed pyrazinediones (2.5%) and numerous unidentified components. After acidification to pH 1, 0.78 g of material was recovered, which contained pyrimidine (3.1%), propanoic acid (11.8%), butanoic acid (26.2%), 3-methyl butanoic acid (4.5%), 2-methyl butanoic acid (15.1%), methyl pyrazine (3.1%), ethyl pyrazine (1%), and numerous unidentified components. After adjusting the pH to 14, 0.29 g of material was recovered, which comprised: pyrimidine (14.1%), 2-methyl piperidine (11.5%), methyl pyrazine (12.9%), 2,5-dimethyl pyrazine (14.8%), trimethyl pyrazine (6%), and numerous unidentified components.

The neutral extract from 5% ZnO gave 0.22 g comprising pyrimidine (4%), cyclopentanone (0.7%), 2-methyl cyclopent-2-en-1-one (4%), methyl pyrazine (12%), 2,5-dimethyl pyrazine (8.8%), 2-ethyl-3-methyl pyrazine (3.6%), trimethyl pyrazine (8.8%), 2-piperidinone (1.6%), 3,6-diisobutyl-2,5-piperazinedione (3.7%), other condensed piperazinediones (1.8%), condensed pyrazinediones (3.3%) and numerous unidentified components. After acidification to pH 1, 0.40 g of material was recovered, which contained pyrimidine (1.6%), 2-methyl cyclopent-2-en-1-one (3.7%), butanoic acid (21.2%), 3-methyl butanoic acid (3%), 2-methyl butanoic acid (10.7%), 4-methyl pentanoic acid (8%), methyl pyrazine (5.1%), 2,5-dimethyl pyrazine (1.4%), and numerous unidentified components. After adjusting the pH to 14, 0.40 g of material was recovered, which comprised: 2-methyl cyclopent-2-en-1-one (1.3%), pyrimidine (0.9%), 4-methyl piperidine (9.4%), methyl pyrazine (5.5%), 2,5-dimethyl pyrazine (5.3%), 2-ethyl-3-methyl pyrazine (1.9%), trimethyl pyrazine (5.2%), 2-piperidinone (4.7%) 3,6-diisobutyl-2,5-piperazinedione (6.8%) and numerous unidentified components.

The neutral extract from 2.5% ZnO gave 1.45 g comprising pyrimidine (2.3%), 2-methyl cyclopent-2-en-1-one (3.6%), methyl pyrazine (11.4%), 2,5-dimethyl pyrazine (9.7%), ethyl pyrazine (11.3%), 2-ethyl-3-methyl pyrazine (3.8%), trimethyl pyrazine (11.2%), 2-ethyl-3,6-dimethyl pyrazine (5.7%), 3,6-diisobutyl-2,5-piperazinedione (3.5%), other condensed pyrazinediones (1.8%) and numerous unidentified components. After acidification to pH 1, 0.52 g of material was recovered, which contained pyrimidine (0.2%), butanoic acid (19.5%), 3-methyl butanoic acid (3.4%), 2-methyl butanoic acid (9%), pentanoic acid (4.2%), 4-methyl pentanoic acid (11.8%), benzene propanoic acid (6.7%), methyl pyrazine (2.3%), ethyl pyrazine (1.2%), 2-piperidinone (4.3%), and numerous unidentified components. After adjusting the pH to 14, 0.25 g of material was recovered, which comprised: pyrimidine (2.1%), 2-methyl piperidine (8.7%), methyl pyrazine (3.9%), 2,5-dimethyl pyrazine (3.1%), trimethyl pyrazine (1.7%), 2-piperidinone (5.5%) 3,6-diisobutyl-2,5-piperazinedione (3.4%) and numerous unidentified components.

### Example 7

### The Addition of Sodium Carbonate

A samples of microalgae (18.6 g/300 mL) was treated with 1.8 g of sodium carbonate and heated to 250° C. The resultant mixture filtered indifferently and gave a yield of solids of 4.92 g.

A sample of the solvent was evaporated to dryness, and to give a solids content of 10.7 g The estimated yield of water-soluble non-volatile organic material was 8.7 g. The aqueous solution was extracted with methylene chloride, then acidified and re-extracted, then made basic and extracted. The components of the extract were:

The neutral extract gave 0.53 g comprising 2-methyl cyclopent2-en-1-one (2%), pyrimidine (1.8%), methyl pyrazine (6.1%), 2,5-dimethyl pyrazine (5.8%), ethyl pyrazine (4.5%), 2-ethyl-3-methyl pyrazine (3.6%), trimethyl pyrazine (7.1%), 2-ethyl-3,6-dimethyl pyrazine (3.8%), 2-piperidinone (3.7%), indole (0.7%), and numerous unidentified components. After acidification to pH 1, 0.52 g of material was recovered, which contained pyrimidine (1.1%), butanoic acid (16%), 3-methyl butanoic acid (4.2%), 2-methyl butanoic acid (8.7%), 4-methyl pentanoic acid (8.1%), benzenepropanoic acid (4.3%), trimethyl pyrazine (2.3%), 2-piperidinone (5.2%) and numerous unidentified components. After adjusting the pH to 14, 0.19 g of material was recovered, which comprised: pyrimidine (2.1%), 2-methyl piperidine (17.6%), methyl pyrazine (7.8%), N-ethyl piperidine (6.4%), 2,5-dimethyl pyrazine (5.6%), trimethyl pyrazine (5.3%), and numerous unidentified components.

### Example 8

### The Addition of Ferric Oxide

A sample of microalgae (18.6 g/300 mL) was treated with 1.8 g ferric oxide and heated to 250° C. A precipitate of microalgae was collected that was filtered with difficulty. The yield of solids was 8.1 g. A sample of the solvent was evaporated to dryness, and the solid content of the solution was 9.5 g. The estimated yield of water-soluble non-volatile organic material was 8.8 g. For each sample, the aqueous solution was extracted with methylene chloride, then acidified and re-extracted, then made basic and extracted. The components of the extract were:

The neutral extract gave 0.81 g comprising xylene (0.6%), nonane (0.9%), undecane (1.6%), 2-methyl cyclopent-2-en-1-one (2.4%), methyl pyrazine (7.5%), 2,5-dimethyl pyrazine (14.8%), trimethyl pyrazine (7.7%), 2-ethyl-3,6-dimethyl pyrazine (3.6%), 2-piperidinone (0.9%), 3,6-diisobutyl-2,5-piperazinedione (2.5%), other condensed piperazinediones (1%), condensed pyrazinediones (2.4%) and numerous unidentified components. After acidification to pH 1, 0.45 g of material was recovered, which contained nonane (0.2%), undecane (0.6%), pentadecene (0.8%), 2-methyl propanoic acid (2.8%), butanoic acid (20.3%), 2-methyl butanoic acid (9%), pentanoic acid (3.5%), 4-methyl pentanoic acid (9.8%), benzene propanoic acid (5.8%), oleic acid (1.2%), 2,5-dimethyl pyrazine (0.5%), 2-piperidinone (3.9%), 3,6-diisobutyl-2,5-piperazinedione (1.1%) and numerous unidentified components. After adjusting the pH to 14, 0.25 g of material was recovered, which comprised: xylene (1%), nonane (2%), decane (3.9%), undecane (1.3%), 2-methyl piperidine (8.5%), methyl piperidine (8.2%), methyl pyrazine (2.7%), 2,5-dimethyl pyrazine (1.5%), and numerous unidentified components.

### Example 9

### Precipitation of Ferric Hydroxide Onto Microalgae

A sample of microalgae (18.6 g/300 mL) was treated with ferric sulphate at 10% concentration, followed by the addition of sufficient ammonia solution to make the pH equal to 8.0 and heated to 250° C. A precipitate of microalgae was collected that was filtered with difficulty. The yield of solids was 8.5 g. A sample of the solvent was evaporated to dryness, and the solid content of the solutions for the solution was 7.2 g. The estimated yield of water-soluble non-volatile organic material was 6.4 g. For each sample, the aqueous solution was extracted with methylene chloride, then acidified and re-extracted, then made basic and extracted. The components of the extract were:

The neutral extract gave 0.23 g comprising undecane (0.6%), 2-methyl cyclopent-2-en-1-one (2.2%), pyrimidine (0.4%), methyl pyrazine (4.4%), 2,5-dimethyl pyrazine (4.6%), ethyl pyrazine (1.8%), trimethyl pyrazine (5.2%), 2-ethyl-3-methyl pyrazine (2.1%), 2-piperidinone (4.3%), condensed piperazinediones (2.7%), condensed pyrazinediones (10.4%) and numerous unidentified components. After acidification to pH 1, 0.32 g of material was recovered, which contained nonane (0.4%), decane (2.5%), undecane (0.6%), cyclopentanone (0.5%), 2-methyl cyclopent-2-en-1-one (2.3%), butanoic acid (22.1%), 2-methyl butanoic acid (12.4%), 4-methyl pentanoic acid (5.8%), methyl pyrazine (6.5%), 2,5-dimethyl pyrazine (3.7%), trimethyl pyrazine (2.3%), 3,6-diisobutyl-2,5-piperazinedione (0.3%) and numerous unidentified components. After adjusting the pH to 14, 0.13 g of material was recovered, which comprised: nonane (0.4%), decane (0.8%), undecane (0.4%), 2-methyl cyclopent-2-en-1-one (0.8%), 2-methyl piperidine (11.8%), methyl pyrazine (4.3%), 2,5-dimethyl pyrazine (4.2%), trimethyl pyrazine (4.1%), 2-piperidinone (6.9%), 3,6-dilsobutyl-2,5-piperazinedione (6.3%) and numerous unidentified components.

### Example 10

### The Addition of Copper Oxide

A sample of microalgae (18.6 g/300 mL) was treated with 1.8 g cupric oxide and heated to 250° C. A precipitate of microalgae was collected that was filtered with difficulty. The yield of solids was 4.8 g, and there was a sign of copper precipitation. A sample of the solvent was evaporated to dryness, and the solid content of the solution was 6.9 g. The estimated yield of water-soluble non-volatile organic material was 6.3 g. For each sample, the aqueous solution was extracted with methylene chloride, then acidified and re-extracted, then made basic and extracted. The components of the extract were:

The neutral extract gave 0.21 g comprising nonane (0.5%), decane (0.9%), undecane (0.7%), 2-methyl cyclopent-2-en-1-one (2.0%), pyrimidine (1%), methyl pyrazine (4.4%), 2,5-dimethyl pyrazine (4.1%), ethyl pyrazine (1.8%), trimethyl pyrazine (4.0%), 2-ethyl-3,6-dimethyl pyrazine (2%), trimethyl hydantoin (0.4%), 2-piperidinone (4.5%), condensed piperazinediones (2.6%), condensed pyrazinediones (9.7%) and numerous unidentified components. After acidification to pH 1, 0.31 g of material was recovered, which contained nonane (0.6%), decane (2.2%) undecane (0.5%), butanoic acid (24.8%), 2-methyl butanoic acid (≈16%), 4-methyl pentanoic acid (5.8%), methyl pyrazine (6.7%), ethyl pyrazine (2.6%), 3,6-diisobutyl-2,5-piperazinedione (0.2%) and numerous unidentified components. After adjusting the pH to 14, 0.11 g of material was recovered, which comprised: nonane (0.7%), decane (3.2%), undecane (1.1%), 2-methyl piperidine (10.5%), methyl pyrazine (1.6%), 2,5-dimethyl pyrazine (1.1%), ethyl pyrazine (1.3%), trimethyl pyrazine (2.1%), 3,6-diisobutyl-2,5-piperazinedione (0.7%) and numerous unidentified components.

### Example 11

### The Addition of Magnesium Oxide

Samples of microalgae (18.6 g/300 mL) were treated with different concentrations of magnesium oxide and heated to 250° C. In each case a clean precipitate of microalgae was collected that was easily filtered and was able to be washed, with 5% magnesium oxide being as good as 10% calcium hydroxide. The yields of solids were: 250° C., 10% MgO, (8.7 g); 5% MgO, (7.9 g); 2.5% MgO (6.2 g), 1.25% MgO (5.81 g). A sample of the solvent was evaporated to dryness, and the solid content of the solutions were 10% MgO, (9.9 g); 5% MgO, (6.5 g), 2.5% MgO (6.3 g). The estimated yield of water-soluble non-volatile organic material was 10% MgO, (9.8 g); 5% MgO, (6.9 g), 2.5% MgO (5.7 g), 1.25% MgO (7.7 g). For each sample, the aqueous solution was extracted with methylene chloride, then acidified and re-extracted, then made basic and extracted. The components of the extract were:

The neutral extract from 10% MgO gave 0.47 g comprising dimethyl disulphide (4.6%), nonane (0.6%), undecane (0.9%), cyclopentanone (0.4%), 2-methyl cyclopent-2-en-1-one (1.9%), methyl pyrazine (11.3%), 2,5-dimethyl pyrazine (20.2%), 2-ethyl-6-methyl pyrazine (4.5%), trimethyl pyrazine (12.1%), 2-ethyl-3,6-dimethyl pyrazine (5.7%), 3,6-dilsobutyl-2,5-piperazinedione (1%), and numerous unidentified components. After acidification to pH 1, 0.28 g of material was recovered, which contained nonane (0.2%), decane (1.5%), undecane (0.4%), 2-methyl propanoic acid (7.9%), butanoic acid (24.5%), 2-methyl butanoic acid (15.9%), 4-methyl pentanoic acid (11.3%), methyl pyrazine (6.1%), 2,5-dimethyl pyrazine (2.1%), 2-piperidinone (1.5%), 3,6-diisobutyl-2,5-piperazinedione (0.2%), a pyrrolopyrazine dione (0.7%) and numerous unidentified components. After adjusting the pH to 14, 0.07 g of material was recovered, which comprised: xylene (0.5%), nonane (0.9%), decane (1.8%), undecane (1%), pyrimidine (1%), 2-methyl piperidine (≈16%), methyl pyrazine (2.3%), 2,5-dimethyl pyrazine (2.4%), trimethyl pyrazine (1%), 2-piperidinone (5.4%), 3,6-diisobutyl-2,5-piperazinedione (2.4%), a condensed piperazinedione (1.2%), a condensed pyrrolopyrazinedione (1.3%) and numerous unidentified components.

The neutral extract from 5% MgO gave 0.69 g comprising undecane (1%), 2-methyl cyclopent-2-en-1-one (1.9%), pyrimidine (1.1%), methyl pyrazine (5.5%), 2,5-dimethyl pyrazine (5.6%), ethyl pyrazine (3.3%), 2-ethyl-3-methyl pyrazine (2.6%), trimethyl pyrazine (5.3%), 2-ethyl-3,6-dimethyl pyrazine (3.3%), 2-piperidinone (3.8%), condensed pyrrolopyrazinediones (8.9%) a piperazinedione (2.2%), and numerous unidentified components. After acidification to pH 1, 0.52 g of material was recovered, which contained 2-methyl propanoic acid (4%), butanoic acid (6.2%), 2-methyl butanoic acid (6.4%), 3-methyl butanoic acid (2.6%), benzene propionic acid (9.4%), 4-methyl pentanoic acid (11.5%), cyclopentanone (0.3%), 2,3-dimethyl cyclopent-2-en-1-one (0.2%), 2-piperidinone (4.1%), and numerous unidentified components. After adjusting the pH to 14, 0.32 g of material was recovered, which comprised: xylene (0.5%), nonane (0.8%), decane (2.5%), undecane (0.5%), pyrimidine (2%), 2-methyl piperidine (6.1%), N-ethyl piperidine (3.6%), butanamine (8.4%), methyl pyrazine (9.3%), 2,5-dimethyl pyrazine (11.5%), trimethyl pyrazine (5%), 2-piperidinone (5.4%), a condensed piperazinedione (0.7%), and numerous unidentified components.

The neutral extract from 2.5% MgO gave 0.30 g comprising nonane (0.5%) undecane (0.7%), 2-methyl cyclopent-2-en-1-one (2.5%), methyl pyrazine (8.8%), 2,5-dimethyl pyrazine (7.2%), ethyl pyrazine (6.0%), trimethyl pyrazine (6.8%), 2-ethyl-3,6-dimethyl pyrazine (3.6%), 2-piperidinone (0.7%), 3,6-diisobutyl-2,5-piperazinedione (4.7%) other condensed pyrrolopyrazinediones (6.4%) condensed piperazinediones (8%), and numerous unidentified components. After acidification to pH 1, 0.68 g of material was recovered, which contained nonane (0.5%), decane (1.7%), dimethyl disulphide (1%), butanoic acid (21.9%), 2-methyl butanoic acid (9.2%), 4-methyl pentanoic acid (6.4%), pyrimidine (1.2%), methyl pyrazine (4.3%), trimethyl pyrazine (1.6%), and numerous unidentified components. After adjusting the pH to 14, 0.16 g of material was recovered, which comprised: xylene (1.1%), nonane (1.1%), decane (3.6%), undecane (1%), pyrimidine (1.3%), methyl pyrazine (8.9%), 2,5-dimethyl pyrazine (6.7%), trimethyl pyrazine (3.5%), 3,6-diisobutyl-2,5-piperazinedione (0.8%) a condensed pyrrolopyrazinedione (3.2%), and numerous unidentified components.

The neutral extract from 1.25% MgO gave 0.34 g comprising 2-methyl cyclopent-2-en-1-one (2.7%), 2.3-dimethyl cyclopent-2-en-1-one (0.7%), pyrimidine (1.1%), methyl pyrazine (6.0%), 2,5-dimethyl pyrazine (5.8%), ethyl pyrazine (2.5%), trimethyl pyrazine (7.1%), 2-ethyl-3,6-dimethyl pyrazine (4%), 2-piperidinone (3.9%), condensed piperazinediones (1.3%), and numerous unidentified components. After acidification to pH 1, 0.36 g of material was recovered, which contained nonane (1%), decane (1.7%), undecane (0.8%), dimethyl disulphide (1.5%), butanoic acid (12.1%), 2-methyl propanoic acid (7.5%), 3-methyl propanoic acid (3.6%), pyrimidine (1.5%), ethyl pyrazine (1.9%), 2-piperidinone (2.1%), 3,6-diisobutyl-2,5-piperazinedione (5%) a condensed pyrrolopyrazinedione (2.3%), and numerous unidentified components. After adjusting the pH to 14, 0.13 g of material was recovered, which comprised: xylene (0.7%), nonane (1.3%), decane (2.9%), undecane (1%), pyrimidine (7.2%), methyl pyrazine (2.4%), 2,5-dimethyl pyrazine (1.8%), 2-piperidinone (4%), 3,6-diisobutyl-2,5-piperazinedione (1.4%) and numerous unidentified components.

### Example 12

### The Addition of Nickel Oxide

Samples of microalgae (18.6 g/300 mL) were treated with nickel oxide (0.9 g) and heated to 250° C. The resultant solid was difficult to filter but settled readily to give 7.6 g solids. A sample of the solvent was evaporated to dryness, and the solid content of the solutions were (7.6 g). The estimated yield of water-soluble non-volatile organic material was (11.4 g). The aqueous solution was extracted with methylene chloride, then acidified and re-extracted, then made basic and extracted. The components of the extract were:

The neutral solution gave 0.23 g comprising nonane (0.5%), undecane (0.7%), cyclopentanone (0.4%), 2-methyl cyclopent-2-en-1-one (1.9%), pyrimidine (1.4%), 2-methyl pyridine (1.3%), methyl pyrazine (6.1%), 2,5-dimethyl pyrazine (14.6%), trimethyl pyrazine (10.9%), 2-ethyl-3,6-dimethyl pyrazine (4.5%), 3,6-diisobutyl-2,5-piperazinedione (4.5%), pyrrolopyrazine diones (4.1%), a condensed piperazine dione (1.4%) and numerous unidentified components. After acidification to pH 1, 0.52 g of material was recovered, which contained nonane (1.1%), decane (2.3%), undecane (0.5%), 2-methyl propanoic acid (7.5%), butanoic acid (19.2%), 3-methyl butanoic acid (4.5%), 2-methyl butanoic acid (13.1%), 3,6-diisobutyl-2,5-piperazinedione (1.6%), a pyrrolopyrazine dione (1.7%) and numerous unidentified components. After adjusting the pH to 14, 0.12 g of material was recovered, which comprised: nonane (1%), decane (3.5%), undecane (0.6%), pyrrolidine (15.3%), 2-methyl piperidine (9.3%), methyl pyrazine (2%), 2,5-dimethyl pyrazine (1.2%) and numerous unidentified components.

### Example 13

### Hydrothermal Reaction of Recovered Filtrate

0.45 g trisodium phosphate was added to 300 mL of filtrate from the run with 10% calcium hydroxide and this was also heated to 400° C. for 30 minutes, and following cooling, 3.42 g of oil was obtained. The aqueous solution was acidified and further extracted to give 0.75 g of extract. The components of the extracts were:

The oil comprised toluene (5.8%), ethyl benzene (11.4%), xylene 2.2%), styrene (2%), nonane (1.3%), decane (2.2%), undecene (1.1%), undecane (1.7%), p-ethyl phenol (1.9%), dodecane (1.7%), pentadecene (1.2%), pentadecane (0.7%), heptadecane (2%), cyclopentanone (1.2%), 2-methyl cyclopentanone (2%), 2-methylcyclopent-2-en-1-one (2.4%), 2,3-dimethylcyclopent-2-en-1-one (1.8%), methyl pyrazine (1.8%), 2,5-dimethyl pyrazine (1.2%),trimethyl pyrazine (1.8%), N-ethyl 2-pyrrolidinone (4.2%). The extract from the acidified aqueous solution comprised acetic acid (32.4%), propionic acid (5.6%), N-methyl acetamide (2.4%), hexanoic acid (0.4%), 4-methyl pentanoic acid (3.1%), N-methyl 2-pyrrolidinone (8.2%), 2-pyrrolidinone (4.5%), N-methyl succinimide (0.3%), N-ethyl 2-pyrrolidinone (1.6%), 2-piperidinone (6.1%), 3,6-diisobutyl-2,5-piperazinedione (0.4%) and numerous unidentified components.

### Example 13

### Isolation of Fatty Acids

Residue from the filtration of a calcium hydroxide reaction (1 g) was stirred with cold dilute hydrochloric acid for 30 minutes, then the mixture was extracted ×2 with methylene chloride. The methylene chloride solution was dried and the methylene chloride was removed by distillation. The acids were then converted to methyl esters and analysed by GCMS following standard methods to give 100 mg of oil. The FAME mixture comprised methyl esters of:

Butanedioic acid (2.1%), benzenepropanoic acid (4.4%), docosanoic acid (0.8%), tetradecanoic acid (2.2%), pentadecanoic acid (1.9%), 17-methyl octadecanoic acid (1.1%), 4,7,13,16,19-docosahexaenoic acid (2.2%), 9-hexadecenoic acid (7.8%), hexadecanoic acid (17.4%), 8,11,14-docosatrienoic acid (17.6%), 9-octadecenoic acid (5.6%), 9,12-octadecadienoic acid (8%), octadecanoic acid (1.8%) and numerous components that were not unambiguously identified.

### INDUSTRIAL APPLICATION

The method of the invention may be used to produce an algal biomass that is readily separable from water, and can be used to obtain microalgae in a solid form, or in concentrated dispersions of water.

The algal biomass made by the process of this invention is sterilized by the heat treatment and has a less obnoxious smell, thus making it more desirable for uses such as a fertilizer or stock food.

Being able to concentrate an aqueous dispersion of algal biomass permits a reduction in the size of subsequent processing equipment for further processing, such as hydrothermal processing to make biofuels.

The process also permits a certain level of separation of components, thus permitting certain materials such as indoles to be isolated in a relatively pure form.

The foregoing description of the invention includes preferred forms thereof. Modifications may be made thereto without departing from the scope of the invention.

1. A method for producing an algal biomass that is readily separable from water, the method comprising heating an aqueous slurry comprising an algal biomass and water in a pressure vessel at a temperature of about 140° C. to about 300° C. and at a pressure that maintains the water in the liquid phase to produce an algal biomass that is readily concentrated or separated from the resultant aqueous phase by means of filtration, centrifugation or settling.

2. A method for producing an algal biomass that is readily separable from water, the method comprising heating an aqueous slurry comprising an algal biomass, a separation agent comprising a metal oxide or hydroxide, and water in a pressure vessel at a temperature of about 150° C. to about 250° C. and at a pressure that maintains the water in the liquid phase to produce an algal biomass that is readily concentrated or separated from the resultant aqueous phase by means of filtration, centrifugation or settling.

3. The method of claim 1, wherein the aqueous slurry further comprises a separation agent comprising a metal oxide or hydroxide.

4. (canceled)

5. The method of claim 2, wherein the metal oxide or hydroxide is an oxide or hydroxide of magnesium, calcium, strontium, barium, zinc or cadmium, or any combination of any two or more thereof.

6. (canceled)

7. The method of claim 1, wherein the aqueous slurry comprises about 1 to about 80% by weight algal biomass.

8. The method of claim 3, wherein the aqueous slurry comprises 1 to about 30% by weight separation agent.

9. (canceled)

10. (canceled)

11. The method of claim 1, wherein the aqueous slurry is heated for about 1 to about 300 minutes.

12. (canceled)

13. (canceled)

14. (canceled)

15. The method of claim 1, wherein the aqueous phase is subjected to further processing to produce a biofuel, a biofuel precursor or one or more organic chemical products.

16. (canceled)

17. (canceled)

18. (canceled)

19. (canceled)

20. (canceled)

21. The method of claim 1, wherein the aqueous phase, optionally following extraction to remove nitrogenous material, is heated to supercritical temperatures.

22. The method of claim 1, wherein the concentrated aqueous dispersion or solids obtained from the concentrated aqueous dispersion are treated with acid to recover fatty acids essentially free of nitrogenous material.

23. The method of claim 1, wherein the concentrated aqueous dispersion or solids obtained from the concentrated aqueous dispersion are used as stock feed or fertilizer.

24. (canceled)

25. The method of claim 3, wherein the metal oxide or hydroxide is an oxide or hydroxide of magnesium, calcium, strontium, barium, zinc or cadmium, or any combination of any two or more thereof.

26. The method of claim 2, wherein the aqueous slurry comprises about 1 to about 80% by weight algal biomass.

27. The method of claim 2, wherein the aqueous slurry comprises 1 to about 30% by weight separation agent.

28. The method of claim 2, wherein the aqueous slurry is heated for about 1 to about 300 minutes.

29. The method of claim 2, wherein the aqueous phase is subjected to further processing to produce a biofuel, a biofuel precursor or one or more organic chemical products.

30. The method of claim 2, wherein the aqueous phase, optionally following extraction to remove nitrogenous material, is heated to supercritical temperatures.

31. The method of claim 2, wherein the concentrated aqueous dispersion or solids obtained from the concentrated aqueous dispersion are treated with acid to recover fatty acids essentially free of nitrogenous material.

32. The method of claim 2, wherein the concentrated aqueous dispersion or solids obtained from the concentrated aqueous dispersion are used as stock feed or fertilizer.
